(19) United States
(12) Patent Application Publication
(10) Pub. No.: US 2024/0285196 A1
(43) Pub. Date: Aug. 29, 2024

(54) A METHOD AND SYSTEM FOR DETERMINING A PERFUSION METRIC USING DEOXYHEMOGLOBIN AS A CONTRAST AGENT

(71) Applicant: THORNHILL SCIENTIFIC INC, North York (CA)

(72) Inventors: David J. MIKULIS, Oakville (CA); Julien POUBLANC, Toronto (CA); Olivia SOBCZYK, Etobicoke (CA); James DUFFIN, Toronto (CA); Ece Su SAYIN, Windsor (CA); Joseph Arnold FISHER, Thornhill (CA)

(21) Appl. No.: 18/572,452

(22) PCT Filed: Jun. 21, 2022
(86) PCT No.: PCT/IB2022/055769

§ 371 Date: Dec. 20, 2023

Related U.S. Application Data
(60) Provisional application No. 63/213,062, filed on Jun. 21, 2021.

(51) Int. Cl. A61B 5/1455; A61B 5/00

(52) U.S. Cl. CPC A61B 5/14551; A61B 5/0042

(57) ABSTRACT

Hypoxia-induced deoxy hemoglobin concentration ([dOHb]) may be used as a susceptibility contrast agent in subjects. While the maximal rate of generating blood [dOHb] are limited by constraints in pulmonary gas mixing, decreasing dOHb with reoxygenation of the lungs can be accomplished in one breath, resulting in a step reduction in arterial [dOHb] and thereby a step increase in cerebral BOLD signal recorded with MRI. The BOLD signal changes accompanying a step decrease in [dOHb] can be analyzed to calculate cerebral perfusion measures and compare their maps to those obtained using a bolus of a conventional contrast agent, gadolinium, and a conventional analysis requiring the identification of an arterial input function. The two methods provided comparable anatomically-distributed hemodynamic information.

### CROSS-REFERENCE TO RELATED APPLICATION(S)

This application claims the benefit of U.S. provisional application entitled “Susceptibility imaging with resonance magnetic imaging using endogenous deoxyhemoglobin as a contrast agent” having Ser. No. 63/213,062, filed Jun. 21, 2021 and incorporated in its entirety by reference herein.

### FIELD

The present specification is directed to perfusion MRI, and specifically methods and systems for using deoxyhemoglobin as a contrast agent.

### BACKGROUND

Gadolinium-based contrast agents are intravenous drugs used in diagnostic procedures to enhance the quality of magnetic resonance imaging (MRI). Gadolinium-based dynamic susceptibility contrast (DSC) is commonly used to characterize blood flow in subjects with stroke and brain tumors. The blood volume in a particular voxel is calculated based on the arterial input function (AIF) and deconvolution of the decrease in MRI signal intensity. Unfortunately, this method is not entirely accurate because it assumes that the impulse caused by the gadolinium is instantaneous. Furthermore, gadolinium contrast agents are associated with adverse reactions and long-term accumulation in tissues.

### SUMMARY

One aspect is a method for determining perfusion metrics using deoxyhemoglobin as a susceptibility agent. The method includes targeting a first partial pressure of oxygen in arterial blood (PaO₂) in a subject using a sequential gas delivery device for a first duration of time, then targeting a second PaO₂ using the sequential gas delivery device for a second duration of time. While targeting the first and second PaO₂, the method includes measuring a blood oxygen level dependent (BOLD) signal in a voxel of the subject's brain using magnetic resonance imaging (MRI) while targeting the first and second PaO₂. Next, calculate a rate of change for the BOLD signal and fit the rate of change to a gamma variate function. Perfusion metrics can be calculated based on the gamma variate function and the integral of the gamma variate function.

A further aspect is another method for determining perfusion metrics using deoxyhemoglobin as a susceptibility agent. The method includes targeting a first partial pressure of oxygen in arterial blood (PaO₂) in a subject using a sequential gas delivery device for a first duration of time, then targeting a second PaO₂ using the sequential gas delivery device for a second duration of time. While targeting the first and second PaO₂, the method includes measuring a magnetic resonance signal in a voxel of the subject's brain using magnetic resonance imaging (MRI) while targeting the first and second PaO₂. Next, calculate a rate of change for the magnetic resonance signal and its integral and calculate perfusion metrics based on the rate of change and its integral.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

The present disclosure will be described with respect to the figures herein.

FIG. 1 is a block diagram of a system for determining a perfusion metric by using deoxyhemoglobin as a contrast agent.

FIG. 2 is a diagram showing temporal profiles of a deoxyhemoglobin signal.

FIG. 3 is a flowchart of a method for determining a perfusion metric by using deoxyhemoglobin as a contrast agent.

FIG. 4 is a flowchart of another method for determining a perfusion metric by using deoxyhemoglobin as a contrast agent.

FIG. 5 is a graph of experimental results showing [dOHb] and BOLD plotted against time.

FIG. 6 is a graph showing a series of gamma variate functions.

FIG. 7 is a graph showing a series of integrations of gamma variate functions.

FIG. 8A is a graph showing the relationship between a gamma variate function and relative cerebral blood volume (rCBV).

FIG. 8B is a graph showing the relationship between a gamma variate function and mean transit time (MTT) and relative cerebral blood flow (rCBF).

FIG. 9 is a graph showing a method of fitting a BOLD signal to a gamma variate function.

FIG. 10A is a map of a subject's brain showing the selected voxel.

FIG. 10B is a graph showing the MRI signal intensity during three gas challenges.

FIG. 10C is a graph showing the MRI signal intensity during the first gas challenge.

FIG. 10D is a graph showing the MRI signal intensity during the second gas challenge.

FIG. 10E is a graph showing the MRI signal intensity during the third gas challenge.

FIG. 11 is a graph showing the average CNR during four gas challenges.

FIG. 12A is a series of maps showing the BOLD signal change, in grayscale.

FIG. 12B is a series of maps showing the BOLD signal change, in grayscale.

FIG. 12C is a series of maps showing the BOLD signal change, in color.

FIG. 12D is a series of maps showing the BOLD signal change, in color.

FIG. 13A is a graph showing the BOLD signal and SaO₂ in venous and arterial voxels.

FIG. 13B is a graph showing BOLD signal and SaO₂ in GM and WM.

FIG. 14A is a series of maps showing BOLD signal and perfusion metrics for a healthy subject in grayscale.

FIG. 14B is a series of maps showing BOLD signal and perfusion metrics for a healthy subject in color.

FIG. 15A is a series of maps showing BOLD signal and perfusion metrics for a subject with left ICA occlusion, in grayscale.

FIG. 15B is a series of maps showing BOLD signal and perfusion metrics for a subject with left ICA occlusion, in color.

### DETAILED DESCRIPTION

“AIF” herein refers to arterial input function.
“BOLD” or “BOLD imaging” herein refers to blood oxygen level dependent imaging.
“dOHb” herein refers to deoxyhemoglobin.
“[dOHb]” herein refers to deoxyhemoglobin concentration.
“PaO₂” herein refers to partial pressure of oxygen in arterial blood.
“P_{ET}O₂” herein refers to partial pressure of oxygen in end tidal (i.e., end expired) breath.
“PaCO₂” herein refers to partial pressure of carbon dioxide in arterial blood.
“P_{ET}CO₂” herein refers to partial pressure of carbon dioxide in end tidal (i.e., end expired) breath.
“OHDC” herein refers to oxyhemoglobin dissociation curve.
“Gd” herein refers to gadolinium.
“GM” herein refers to gray matter.
“WM” herein refers to white matter.
“MRI” herein refers to magnetic resonance imaging.
“MTT” herein refers to mean transit time.
“rCBV” “herein refers to relative cerebral blood volume.
“rCBF” herein refers to relative cerebral blood flow.
“SₐO₂” herein refers to arterial hemoglobin saturation.
“TFA” herein refers to transfer function analysis.
“TofA” herein refers to blood arrival time.
“TR” herein refers to time of repetition.

Deoxyhemoglobin has been explored as a safer alternative to gadolinium. Deoxyhemoglobin is an endogenous molecule that causes few adverse reactions and does not accumulate in tissues.

BOLD sequences are sensitive to distortions in the static magnetic field caused by the concentration of compartmentalized paramagnetic moieties such as deoxyhemoglobin (dOHb) and gadolinium-based contrast agents. The time constant of the exponential decay of the BOLD signal T′₂ is inversely proportional to the concentration of the paramagnetic moieties with the proviso that compartmentalization is unchanged. Importantly, there are near linear decreases in intravascular T′₂ increasing deoxyhemoglobin concentration [dOHb] at 3 Tesla, similar to the effect seen with gadolinium-based contrast agents.

Deoxyhemoglobin provides a number of advantages over gadolinium as a contrast agent. Firstly, as an endogenous molecule, it is safe to administer and does not cause side effects or significant discomfort to the subject. Deoxyhemoglobin does not accumulate or recirculate because it reverts to oxyhemoglobin after returning to the lungs. Secondly, inspired gas almost instantaneously distributes throughout the lungs with each inspiration, resulting in near instantaneous equilibration with the pulmonary blood volume. Thirdly, it is safe and well-tolerated to target repeated changes in dOHb, so individuals and populations can be studied over time.

Changes in PaO₂ of the subject may be implemented with a sequential gas delivery (SGD) device.

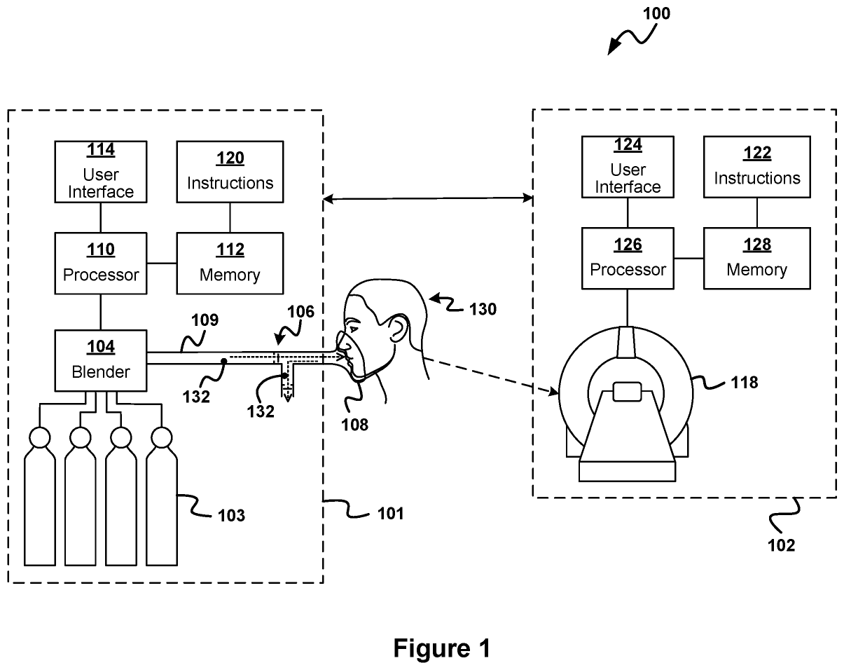

FIG. 1 shows a system 100 for using dOHb as a contrast agent. The system 100 includes a device 101 to provide sequential gas delivery to a subject 130 and target a PₐO₂ while maintaining normocapnia. The system 100 further includes a magnetic resonance imaging (MRI) system 102. The device 101 includes gas supplies 103, a gas blender 104, a mask 108, a processor 110, memory 112, and a user interface device 114. The device 101 may be configured to control end-tidal PCO₂ and end-tidal PO₂ by generating predictions of gas flows to actuate target end-tidal values. The device 101 may be an RespirAct™ device, made by Thornhill Medical™ of Toronto, Canada, specifically configured to implement the techniques discussed herein. For further information regarding sequential gas delivery, U.S. Pat. No. 8,844,528, US Publication No. 2018/0043117, and U.S. Pat. No. 10,850,052, which are incorporated herein by reference, may be consulted.

The gas supplies 103 may provide carbon dioxide, oxygen, nitrogen, and air, for example, at controllable rates, as defined by the processor 110. A non-limiting example of the gas mixtures provided in the gas supplies 103 is:
a. Gas A: 10% O₂, 90% N₂;
b. Gas B: 10% O₂, 90% CO₂;
c. Gas C: 100% O₂; and
d. Calibration gas: 10% O₂, 9% CO₂, 81% N₂.

The gas blender 104 is connected to the gas supplies 103, receives gases from the gas supplies 103, and blends received gases as controlled by the processor 110 to obtain a gas mixture, such as a first gas (G1) and a second gas (G2) for sequential gas delivery.

The second gas (G2) is a neutral gas in the sense that it has about the same PCO₂ as the gas exhaled by the subject 130, which includes about 4% to 5% carbon dioxide. In some examples, the second gas (G2) may include gas actually exhaled by the subject 130. The first gas (G1) has a composition of oxygen that is equal to the target P_{ET}O₂ and preferably no significant amount of carbon dioxide. For example, the first gas (G1) may be air (which typically has about 0.04% carbon dioxide), may consist of 21% oxygen and 79% nitrogen, or may be a gas of similar composition, preferably without any appreciable CO₂.

The processor 110 may control the gas blender 104, such as by electronic valves, to deliver the gas mixture in a controlled manner.

The mask 108 is connected to the gas blender 104 and delivers gas to the subject 130. The mask 108 may be sealed to the subject's face to ensure that the subject only inhales gas provided by the gas blender 104 to the mask 108. In some examples, the mask is sealed to the subject's face with skin tape such as Tegaderm™ (3M, Saint Paul, Minnesota). A valve arrangement 106 may be provided to the device 101 to limit the subject's inhalation to gas provided by the gas blender 104 and limit exhalation to the room. In the example shown, the valve arrangement 106 includes an inspiratory one-way valve from the gas blender 104 to the mask 108, a branch between the inspiratory one-way valve and the mask 108, and an expiratory one-way valve at the branch. Hence, the subject 130 inhales gas from the gas blender 104 and exhales gas to the room.

The gas supplies 103, gas blender 104, and mask 108 may be physically connectable by a conduit 109, such as tubing, to convey gas. Any number of sensors 132 may be positioned at the gas blender 104, mask 108, and/or conduits 109 to sense gas flow rate, pressure, temperature, and/or similar properties and provide this information to the processor 110. Gas properties may be sensed at any suitable location, so as to measure the properties of gas inhaled and/or exhaled by the subject 130.

The processor 110 may include a central processing unit (CPU), a microcontroller, a microprocessor, a processing core, a field-programmable gate array (FPGA), an application-specific integrated circuit (ASIC), or a similar device capable of executing instructions. The processor may be connected to and cooperate with the memory 112 that stores instructions and data.

The memory 112 includes a non-transitory machine-readable medium, such as an electronic, magnetic, optical, or other physical storage device that encodes the instructions. The medium may include, for example, random access memory (RAM), read-only memory (ROM), electrically erasable programmable read-only memory (EEPROM), flash memory, a storage drive, an optical device, or similar.

The user interface device 114 may include a display device, touchscreen, keyboard, buttons, the like, or a combination thereof to allow for operator input and/or output.

Instructions 120 may be provided to carry out the functionality and methods described herein. The instructions 120 may be directly executed, such as a binary file, and/or may include interpretable code, bytecode, source code, or similar instructions that may undergo additional processing to be executed. The instructions 120 may be stored in the memory 112.

System 100 further includes an MRI system 102 for conducting magnetic resonance imaging on the subject 130. A suitable MRI system may include an imaging device such as a 3T MRI system (Signa HDxt-GE Healthcare, Milwaukee). The MRI system 102 may further include a processor 126, memory 128, and a user interface 124. Any description of the processor 126 may apply to the processor 110 and vice versa. Likewise, any description of memory 128 may apply to memory 112 and vice versa. Similarly, any description of instructions 112 may apply to instructions 120 and vice versa. Also, any description of user interface 124 may apply to user interface 114, and vice versa. In some implementations, the MRI system 102 and the device 101 share one or more of a memory, processer, user interface, and instructions, however, in the present disclosure, the MRI system 102 and the device 101 will be described as having respective processors, user interfaces, memories, and instructions. The processor 110 of the device 101 transmits data to the processor 126 of the MRI system 102. The system 100 may be configured to synchronize MRI imaging obtained by the MRI system 102 with measurements obtained by the device 101.

The processor 126 may retrieve operating instructions 122 from the memory or may receive operating instructions 122 from the user interface 124. The operating instructions 122 may include image acquisition parameters. The parameters may include an interleaved echo-planar acquisition consisting of a number of contiguous slices, a defined isotropic resolution, a diameter for the field of view, a repetition time, and an echo time. In one implementation, the number of contiguous slices is 27, the isotropic resolution is 3 mm, the field of view is 19.6 cm, the echo time is 30 ms, and the repetition time (TR) is 2000 ms, however a range of values will be apparent to a person of ordinary skill in the art. The operating instructions 122 may also include parameters for a high-resolution T1-weighted SPGR (Spoiled Gradient Recalled) sequence for co-registering the BOLD images and localizing the arterial and venous components. The SPGR parameters may include a number of slices, a dimension for the partitions, an in-plane voxel size, a diameter for the field of view, an echo time, and a repetition time. In one implementation, the number of slices is 176 m, the partitions are 1 mm thick, the in-plane voxel size is 0.85 by 0.85 mm, the field of view is 22 cm, the echo time is 3.06 ms, and the repetition time (TR) is 7.88 ms.

The processor 126 may be configured to analyze the images using image analysis software such as Matlab 2015a and AFNI (Cox, 1996) or other processes generally known in the art. As part of the analysis, the processor 126 may be configured to perform slice time correction for alignment to the same temporal origin and volume spatial re-registration to correct for head motion during acquisition. The processor 126 may be further configured to perform standard polynomial detrending. In one implementation, the processor 126 is configured to detrend using AFNI software 3dDeconvolve to obtain detrended data.

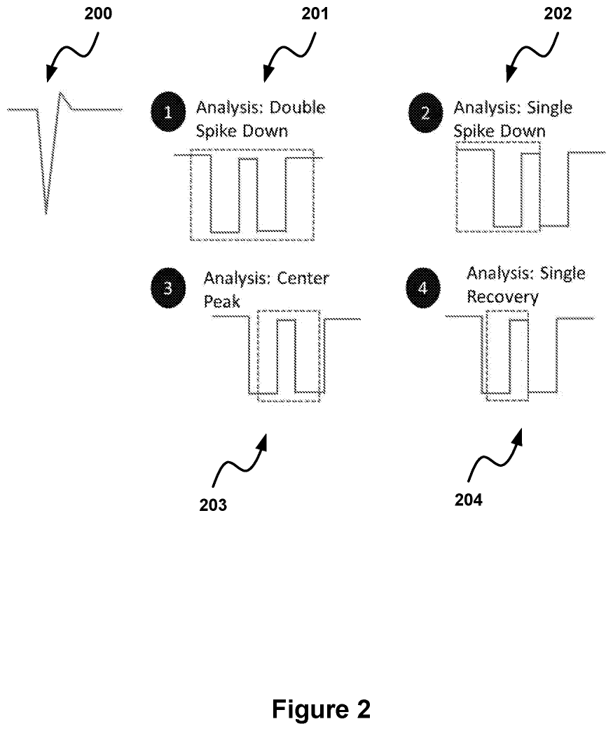

As shown in FIG. 2, the system 100 may be used to implement signals with various temporal profiles.

In one example, the system 100 may be programmed to administer a transient [dOHb] susceptibility bolus in ways that mimic a pattern of susceptibility following an injection of a gadolinium bolus. An example of this signal is shown at 200. However, the measure of hemodynamic parameters from a residue function requires the assumption of an impulse input function. Practically, this cannot be physically obtained from an intravenous injection of contrast or a similar transient change in [dOHb]. As such, the actual obtainable arterial input function (AIF) must be identified and deconvolved back to a virtual impulse input function. All errors in identifying a suitable AIF, and in the assumptions and calculations to simulate an impulse input function, must be subsumed in hemodynamic parameters calculated on that basis.

The present disclosure addresses this limitation by using the system 100 to administer one or more “step change”. In this approach, the system 100 targets a first partial pressure of oxygen in arterial blood (PaO₂) and then target a second PaO₂. Rather than implementing a transient spike, the second PaO₂ is maintained for a duration of time. The device may alternate between targeting the first and second PaO₂ any suitable number of times. Furthermore, the first PaO₂ may be higher or lower than the second PaO₂. One example of a step change pattern is shown at 201. In pattern 201, the system 100 targets a first PaO₂, decreases to a second PaO₂, increases to the first PaO₂, decreases to the second PaO₂, and increases to the first PaO₂. In pattern 202, the system 100 targets a first PaO₂, decreases to a second PaO₂, and increases to the first PaO₂. In pattern 203, the system 100 targets a first PaO₂, increases to a second PaO₂, and decreases to the first PaO₂. In pattern 204, the system targets a first PaO₂ and increases to a second PaO₂.

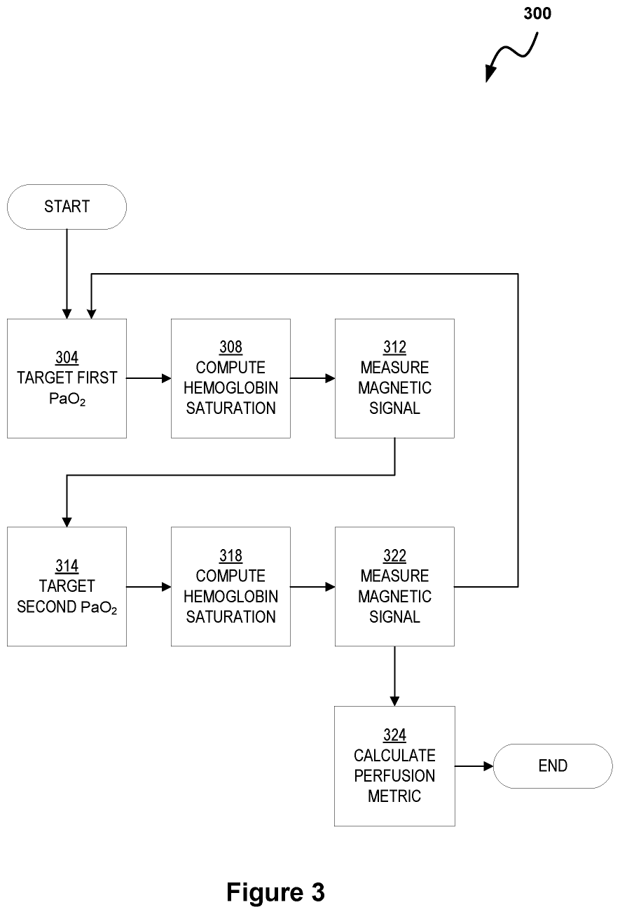

FIG. 3 shows an example method 300 of administering a deoxyhemoglobin signal in a subject using SGD and calculating a perfusion metric. The method 400 may be implemented by instructions 120 and/or instructions 122.

At block 304, the instructions 130 control the device 101 to target the first PaO₂. The device 101 targets the first P_{ET}O₂ for a first duration.

At block 308, and while the device 101 is targeting the first PaO₂, the instructions 120 control the sensor 132 to measure the P_{ET}O₂. Using Equation 1 and the measured PaO₂, the instructions 120 compute the SₐO₂.

$${S}_{a} {O}_{2} = 100 \frac{{K ( {P}_{E T} {O}_{2} )}^{n}}{1 + {K ( {P}_{E T} {O}_{2} )}^{n}} \qquad Equation 1$$

In one implementation of Equation 1, n=−4.4921 pH, K=5.10⁻¹⁴² pH^{157.31}, and pH=7.4. [dOHb]=[dOHb]×(1−SaO₂).

At block 312, the device 102 measures a first magnetic signal in a voxel in the subject's brain.

At block 314, the instructions 120 control the device 101 to target a second PaO₂ for a second duration. At block 318, and while the device 101 is targeting the second PaO₂, the instructions 120 control the sensor 132 to measure the P_{ET}O₂ at block 218. Using Equation 1 and the measured PaO₂, the instructions 120 compute the SₐO₂.

At block 322, the device 102 measures a second magnetic signal in the voxel in the subject's brain.

From block 322, the method 200 may return to block 204 and repeat the subsequent steps. The method may be repeated any suitable number of times.

Lastly, at block 324, the processor 120 computes a perfusion metric based on the first and second magnetic signals. First the processor 120 computes ΔBOLD based on the difference between the first and second magnetic signals.

At block 324, arterial and venous voxels can be extracted using the correlation using ΔBOLD, (correlation coefficient) R, and time delay (TD). In a non-limiting example, arterial voxels are defined as all voxels with ΔBOLD>20%, R>0.8 and 0<TD<1.5 see, and venous voxels are defined as all voxels with ΔBOLD>20%, R>0.8 and 3<TD<5. BOLD time series may be averaged within the arterial and venous components.

Assuming a linear relationship between S_{c,t} and [dOHb], standard tracer kinetic modeling may be used to calculate MTT and relative CBV (rCBV) as per:

$${S}_{c , t} = \frac{r C B V}{M T T} \times AIF ⊗ {R}_{t} + {β}_{1} \times t + {β}_{2} + {ε}_{t} \qquad Equation 2$$

In Equation 2, β₁ and β₂ account for linear signal drift and baseline, respectively. εₜ represents the residuals. SaO₂ was used as the arterial input function (AIFₜ).

$$R t = {e}^{- \frac{t}{M T T}}$$

is the residue function. It is equal to 1 at time 0 and may be set to 0 at time equal to 5×MTT. MTT may be bound between 1 and 8 s. rCBV and MTT may be determined using a least square fitting procedure. The processor 102 can then recalculate rCBF using the central volume theorem as: rCBF=rCBV/MTT. rCBV and rCBF may be respectively multiplied by 15 and 750 to obtain easily readable values within range of absolutes measures.

Assuming that the chosen voxel over the middle cerebral artery (MCA), the AIF, contained 100% blood, absolute CBV in mL/100 g may be calculated according to Equation 3.

$$C B V = 100 ( \frac{kH}{ρ} ) ( \frac{r C B V}{r C B {V}_{AIF}} ) \qquad Equation 3$$

In one example, with tissue density: ρ=1.04 g/cc and difference in hematocrit in large vessels and capillaries: kH=0.73. CBF in mL/100 g/min may be then calculated according to Equation 4:

$$C B V = \frac{C B F}{M T T} \qquad Equation 4$$

The T1-weighted images may be segmented into gray matter (GM), white matter (WM) and cerebrospinal fluid using SPM8 software (Ashburner, J. & Friston, K. J. Neuroimage (2005). The probability density maps obtained may be thresholded at 0.8 to generate a gray matter (GM) and white matter (WM) masks. One layer of peripheral voxels was eroded from the WM mask. GM and WM masks were used to calculate average values for DBOLD signal, CNR, TD, MTT, rCBV and rCBF, CBV and CBF.

The processor 120 may be configured to use Matlab 2015a and AFNI (Cox RW, Comput. Biomed. Res. (1996)) to process the magnetic signals obtained at blocks 312 and 322. First, standard pre-processing steps may include slice time correction and volume re-registration. SaO₂ may be resampled and interpolated to TR intervals, and time-aligned to one voxel placed over the MCA. This alignment may be carefully chosen to have the onset rise of SaO2 and MCA signal match. The baseline mean (S₀) may be calculated over about 30 seconds prior to the rise of SaO2. BOLD signal Sₜ may be scaled according to Equation 5.

$${S}_{c , t} = \frac{{S}_{t} - {S}_{0}}{{S}_{0}} \qquad Equation 5$$

Subscript t indicates that the variable is a function of time. A time delay (TD) may be calculated using cross-correlation between S_{c,t} and multiple SaO₂ curves time shifted from 0 to 7 seconds by intervals of 0.2 seconds. The time shift needed to obtain maximum correlation (R) with S_{c,t} may be extracted for each voxel to generate a TD. S_{c,t} may be regressed against the voxel-wise shifted SₐO_{2,t}^{shifted} to calculate the slop of regression, as shown in Equation 6:

$${S}_{c , T} = α \times {S}_{a} {O}_{2 , t}^{shifted} + {β}_{1} \times t + {β}_{2} + {ε}_{t} \qquad Equation 6$$

α is the slope of the regression, β₁ and β₂ account for respectively linear signal drift and baseline. εₜ represents the residuals.

Using the slope of the regression α, BOLD signal change (ΔBOLD) and CNR may be computed according to Equation 8:

$$Δ BOLD = α ( max ( {S}_{a} {O}_{2 , t}^{shfted} ) - min ( {S}_{a} {O}_{2 , t}^{shfted} ) ) \qquad Equation 7$$

$$CNR = \frac{Δ BOLD}{std ( {ε}_{t} )} \qquad Equation 8$$

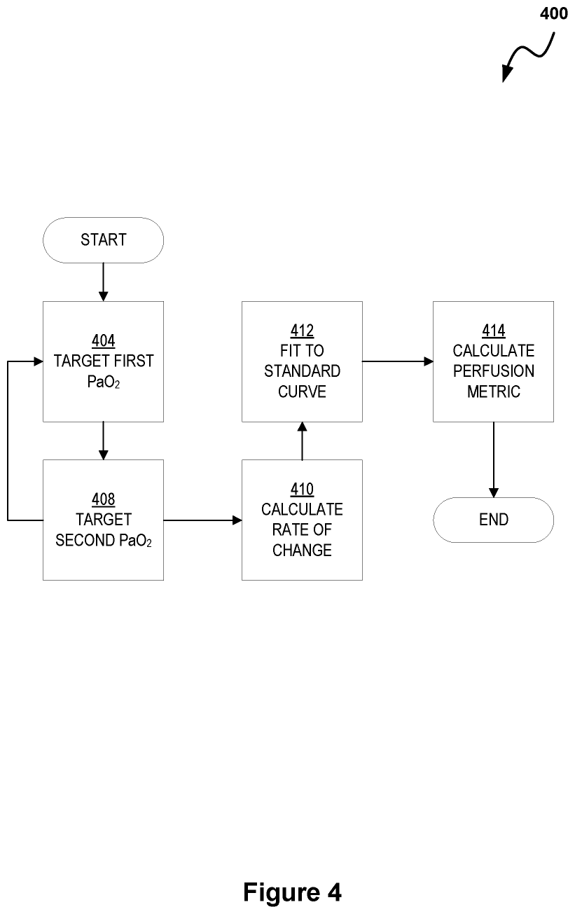

FIG. 4 shows another method 400 of administering a deoxyhemoglobin signal in a subject using SGD and calculating a perfusion metric based on a rate of change in a magnetic resonance signal. The method 400 may be implemented by instructions 120 and/or instructions 122. In method 400, the system 100 implements a step change in the [dOHb] in the subject, which allows the method to overcome several drawbacks.

Methods which compute hemodynamic parameters from a residue function, require the assumption of an impulse input function. Practically, this cannot be physically obtained from an intravenous injection of contrast or a similar transient change in [dOHb]. As such, the actual obtainable arterial input function (AIF) must be identified and deconvolved back to a virtual impulse input function. All errors in identifying a suitable AIF, and in the assumptions and calculations to simulate an impulse input function, must be subsumed in hemodynamic parameters calculated on that basis.

Applying a step change signal addresses the limitations imposed on the measure of hemodynamic parameters by the transient change in susceptibility that is characteristic of the administration of an intravenous bolus of contrast agent, or a transitory change in PaCO₂. T

The imaging data obtained from a step change in BOLD signal is more representative of hemodynamic status as it is more directly measured. Since AIF is not required, method 400 avoids potential errors made in the course of identifying the AIF and back calculating a step input function. The signal transition can be fit with a function whose derivative is a gamma variate in order to solve for the hemodynamic parameters. The hemodynamic parameters obtained through the present method are comparable to measures generated using an AIF from a bolus change of gadolinium.

At block 404, the instructions 120 control the device 101 to target the first PₐO₂ value in the subject. The first PₐO₂ value may correspond with hypoxia, normoxia, or hyperoxia. The device 101 may target the first PaO2 value for any suitable duration of time. The first targeted PaO₂ may be between 20 and 20 mmHg.

At block 408, the instructions 120 control the device 101 to target the second PₐO₂ value in the subject. The first PaO₂ is either higher or lower than the second PaO₂. The second PaO₂ value may correspond with hypoxia, normoxia, or hyperoxia. The device 101 may target the first PaO₂ value for any suitable duration of time. The first and second targeted PaO₂ may be between 20 and 120 mmHg.

In a non-limiting example, the first PaO₂ is about 20 mmHg and the second PaO₂ is about 95 mmHg.

In some examples, the device 101 targets the second PaO₂ within one breath by the subject, causing the subject's PaO₂ to transition from the first PaO₂ to the second PaO₂ within one breath. More accurate results may be obtained if the instructions 120 control the device 101 to target the second PaO₂ within a short period of time. For this reason, it may be advantageous to select a first PaO₂ that is lower than the second PaO₂. Sequential gas delivery techniques, such as the ones described above, can raise PaO₂ more quickly than they can reduce PaO₂.

At blocks 404 and 408, the instructions 120 prospectively target the first or second pressure of oxygen in the arterial blood of the subject (PaO₂) by controlling the device 101 to deliver a first volume of a first gas (G1) to the subject 130 over a first portion of an inspiration by the subject 130. The first volume is selected to be less than or equal to an estimated or expected alveolar volume (V_{A}) of the subject 130 when the subject is breathing normally. The first gas (G1) has a PO₂ that is calculated to result in the targeted PaO₂ after inspiration, taking into account the exchange in O₂ with the perfusing capillary blood and a concentration of CO₂ calculated to result in target lung P_{CO}PCO₂₂ after inspiration. The instructions 120 deliver a second volume of a second, neutral gas (G2) to the subject 130 over a second portion of the inspiration. The second gas is a neutral gas that has a PCO₂ and PO₂ corresponding to the targeted PCO₂ and PO₂ in the exhaled gas respectively. The available volume of second gas (G2) is unlimited in the sense that during normal or deep breathing, the end of the inspiration will contain as much second gas (G2) as needed.

Blocks 404 and 408 may repeated any suitable number of times to acquire more data and improve the accuracy of the outputs. In a non-limiting example, the instructions 120 target the first PaO₂ for 60 seconds and target the second PaO₂ for 20 seconds. This may be repeated any number of suitable times. In some implementations, the instructions 120 may start and end with targeting the first PaO₂.

The instructions 120 may cause the device 101 to target the first PaO₂ for a first duration then target the second PaO₂ for a second duration. The instructions 120 may measure the durations in breaths by the subject or in seconds and minutes. In some examples, the subject 130 is directed to breathe at a pre-determined frequency. In a non-limiting example, the subject 130 is directed to breathe at 30 breaths per minute. The first and second durations of time may be 10 seconds, 20 seconds, 40 seconds, 60 seconds, or any suitable number. The first and second durations may be equal or different in length. Durations and granularity will vary with breathing frequency. The breathing rate of the subject 130 may be controlled and changed so as to individualize the granularity and precision of durations of stimulus and baseline. If either the first or second PaO₂ corresponds to hypoxia, the respective duration may be less than 3 minutes to reduce the effect of hypoxia on blood flow and minimize discomfort experienced by the subject. Since blood flow increases approximately 3 minutes after the onset of hypoxia, the instructions 120 may be programmed to return the subject 130 to normoxia within 3 minutes.

As part of blocks 404 and 408, the instructions 120 may cause the sensors 132 to measure a PO₂ at the end of an exhalation by the subject 130 occurring after delivery of the first and second gases (G1, G2), also known as the end tidal partial pressure of oxygen (P_{ET}O₂). The sensors 132 may measure the P_{ET}O₂ after every breath or every second breath. Since with sequential gas delivery of a first gas and second neutral gas, gases in the lung equalize with gases in the arterial blood, P_{ET}O₂ is approximately equal to PaO₂, and the two terms may be used interchangeably throughout the present specification. In some examples, the instructions 120 control the device 101 to target the first PaO₂ until the sensors 132 detect that the first PaO₂ has been reached. Subsequently, the instructions 120 may control the device 101 to target the second PaO₂ until the sensors 132 detect that the second PaO₂ has been reached. In other examples, the instructions 120 control the device 101 to target the first PaO₂ for a pre-determined duration of time and then target the second PaO₂ for a pre-determined duration of time.

The measured P_{ET}O₂ values may be stored in memory 112. The processor 110 may convert the measured P_{ET}O₂ values into SₐO₂ or [dOHb] values. The processor 110 may calculates the arterial hemoglobin saturation (SₐO₂) based on the measured P_{ET}O₂ values by applying the Hill equation (shown above at Equation 1) or an equivalent method. In Equation 1, the dissociation constant (K) and the Hill coefficient (n) are determined using methods described in Balaban et al., 2013.

As the device 101 is performing block 404 and 408, the MRI system 102 conducts magnetic resonance imaging on the subject 130, as represented at block 210. The MRI system 102 measures a magnetic resonance signal in a voxel in the subject's brain. The magnetic resonance signal can be measured incrementally or continuously as the device 101 targets the first and second PaO₂ values in the subject. For example, the MRI system 102 may incrementally measure the magnetic resonance value every 0.01-10 seconds. In particular, the MRI system 102 measures a magnetic resonance signal as the subject's actual PaO₂ transitions from the first to the second PₐO₂. In some examples, the magnetic resonance signal is a blood oxygen dependent (BOLD) signal.

As part of block 410, the magnetic resonance signals acquired by the MRI system may be stored in memory 128. The processor 126 may analyze the magnetic resonance signals. The processor 126 may cross-correlate the magnetic resonance signal with the SaO₂ or [dOHb] values obtained at blocks 404 and 408.

At block 412, the processor 126 fits the magnetic resonance signal (obtained at block 408) to a standard curve. The fit is based on the rate of change in the magnetic signal acquired at block 410. One example of a standard curve is a function whose integral is a gamma variate (referred to herein as a “gamma variate function”).

In examples where the standard curve is a gamma variate function, the processor 120 calculates the derivative of the magnetic signal and then selects a gamma variate that fits the derivative of the magnetic signal. The processor 120 computes the integral of the selected gamma variate.

At block 414, the processor 120 uses the fit to calculate a perfusion metric for the voxel. The processor 120 may calculate the perfusion metric for the voxel based on either the gamma variate or the integral of the gamma variate function. The calculation is based on the gamma variate function at block 412. For example, the rCBV may be=the area over above the curve for the gamma variate function. In another example, the half-amplitude width of the gamma variate is equal to the MTT. In a further example, the rCBF is equal to the maximum rate of magnetic signal increase, as represented by the amplitude of the gamma variate.

### Example 1

### 1.1: Participant and Ethics Approval

This study conformed to the standards set by the latest revision of the Declaration of Helsinki and was approved by the Research Ethics Board of the University Health Network (UHN) and Health Canada. Written informed consent to partake in this study was obtained from all participants. Healthy control volunteers (HC group) were recruited by word of mouth. They were non-smokers, not on any medication and had no known history of neurological or cardiovascular disease. After their scan acquisition all participants', results were examined by a neuroradiologist (DJM) for white matter hyperintensities and strokes to ensure the selection of participants without such complications. Subjects were recruited from the outpatient hematology clinic at UHN.

### 1.2: Application of MR Contrast

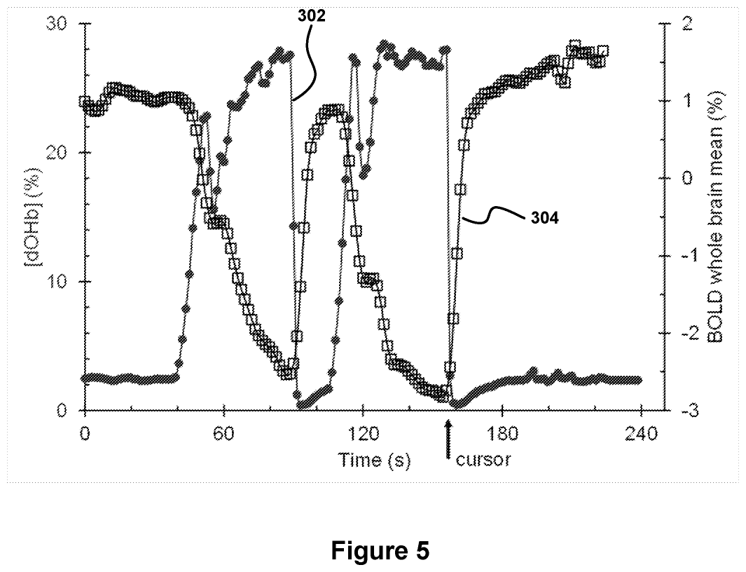

The [dOHb] changes were achieved by controlling P_{ET}O₂ and P_{ET}CO₂ using sequential delivery of inspired gases with a computer-controlled gas blender (RespirAct™; Thornhill Medical Inc, Toronto, Canada) running a prospective targeting algorithm. Participants breathed through a facemask sealed to the face with skin tape, specifically Tegaderm™ (3M™, Saint Paul, MN, U.S.A.), to exclude all but system-supplied gas. The programmed P_{ET}O₂ stimulus pattern was 4-minutes and 20 seconds long and is shown in FIG. 5. After the completion of the P_{ET}O₂ sequence, the participant returned to free breathing on room air for at least 5 minutes before the Gd based perfusion acquisition, which consisted of an intravenous injection of Gadovist®, 5 ml at a rate of 5 ml/s followed by 30 ml of saline at a rate of 5 ml/s.

FIG. 5 shows the hypoxia-induced changes in [dOHb] (%) at 302 and the resulting whole brain average BOLD (%) signal response at 304 in a representative participant. [dOHb] was calculated from P_{ET}O₂ using the Hill equation (Equation 1) describing the normal oxyhemoglobin in-vivo O₂ dissociation curve. The black cursor arrow is placed at the start of the BOLD step change and the [dOHb] is aligned to it. The respiratory paradigm consisted of a 60 second normoxic baseline P_{ET}O₂ of 95 mmHg, a hypoxic step of P_{ET}O₂ to 40 mmHg for 60 seconds, a return to normoxia for 20 seconds, a second hypoxic step for 60 seconds, followed by a return to normoxia for 60 seconds. Each data point on the [dOHb] axis and each data point on the BOLD axis represents one TR (time of reptition).

### 1.3: MRI Scanning Protocol

These experiments were performed in a 3-Tesla scanner such as HDx Signa platform (GE healthcare, Milwaukee, Wisconsin) with an 8-channel head coil. The scanning protocol consisted of a high-resolution T1-weighted scan followed by two BOLD sequence scans. Two BOLD scan sequences were used: The first during P_{ET}O₂ manipulation while maintaining isocapnia at the individual's resting P_{ET}CO₂. The second after at least 5 minutes breathing room air after an intravenous injection of gadolinium. The high-resolution T1-weighted scan was acquired using a 3D spoiled gradient echo sequence with the following parameters: TI=450 ms, TR 7.88 ms, TE=3 ms, flip angle=12º, voxel size=0.859×0.859×1 mm, matrix size=256×256, 146 slices, field of view=24×24 cm, no interslice gap. The BOLD data was acquired using a T2*-weighted gradient echoplanar imaging sequence with the following parameters: TR=1500 ms, TE=30 ms, flip angle=73°, 29 slices voxel size=3 mm isotropic voxels and matrix size=64×64.

### 1.4: Data Analysis

The acquired BOLD images were volume registered, slice-time corrected and co-registered to the anatomical images using AFNI or “Analysis of Functional Neurolmages” software (National Institutes of Health, Bethesda, Maryland). [dOHb] (%) was calculated from end tidal PO₂ using the Hill equation (Equation 1) describing the normal oxyhemoglobin in-vivo O₂ dissociation curve. LabVIEW™ software (National Instruments, Texas) was used to fit the observed step change in BOLD (%) signal for each voxel and perfusion parameters were estimated from these fits. The processing is detailed in section 1.5 (below).

For the gadolinium analysis, FSL tool VERBENA software 23 (University of Oxford, Oxford, United Kingdom) was used to calculate MTT using the gadolinium data protocol and the measured BOLD signal over the middle cerebral artery as the arterial input function (AIF).

Maps of the dOHb and Gd perfusion measures were generated using AFNI software (National Institutes of Health, Bethesda, Maryland). and overlayed onto their respective anatomical images. Analytical processing software, SPM8 (Wellcome Department of Imaging Neuroscience, Institute of Neurology, University College, London, UK), was used to segment the anatomical images (T1 weighted) into gray matter (GM) and white matter (WM) and a threshold of 70% probability was applied. This was done by re-registering all files to the T1 MNI template (MNI152) via a nonlinear transformation and spatially smoothed with a full width at half maximum of 5 mm, to minimize inter subject co-registration errors. Then using SPM8 normalization, images were transformed into Montreal Neurological Institute (MNI) space, using a non-linear re-registration.

Average MTT for GM, and WM were calculated for each participant. The 24-person cohort of healthy adults was used to create an atlas of data of healthy participants to establish normative data for MTT. Results from a 34-person subject group were compared to the 24 healthy participant control group atlas. The participant-specific GM and WM masks generated previously were used to calculate MTT for GM and WM.

The fitted BOLD step function was integrated to provide an area of the change to estimate relative cerebral blood volume (rCBV). The associated derivate gamma variate yielded a half-amplitude full width to estimate mean capillary transit time (MTT) in seconds, and a peak value of the maximum rate of change to estimate relative cerebral blood flow (rCBF) (i.e., blood flow relative to the intravascular volume). Maps of these measures were then compared with those obtained from the analysis of AIF obtained from [dOHb] and Gd.

### 1.5: Fitting the Step Change in BOLD (%) Signal Change to Derive Estimates of Perfusion Parameters

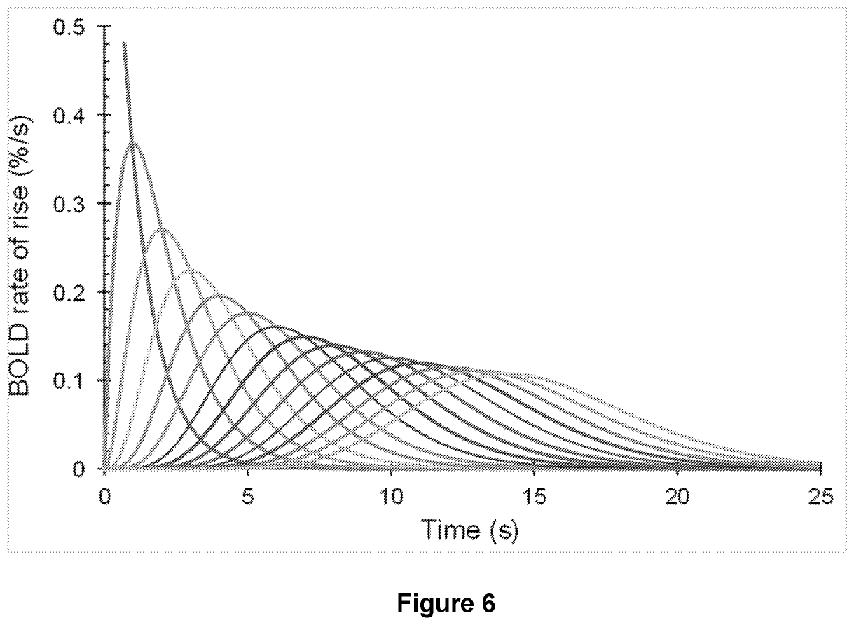

As shown in FIG. 5, a cursor is placed at the start of the step rise in the whole brain mean BOLD signal. This cursor time is then used as the reference start time for all voxels. The rate of rise of the BOLD signal during the step is assumed to have the shape of a gamma variate, as shown in FIG. 6. In this example, the gamma variate function is determined according to Equation 9, where a and b are shape parameters and t is time:

$$\int ( a , b , t ) = \frac{{t}^{a - 1} * {e}^{( - \frac{t}{b} )}}{{b}^{a} * Γ ( a )} \qquad Equation 9$$

The solution may be provided in any suitable units such as percentage per seconds (%/s). FIG. 6 shows the BOLD signal rate of rise (%/s) vs. time (0-25 s) from Equation 9, where a=1, 2 . . . 15; b=1.

This assumption provided a series of shapes for the observed BOLD step change that varies from an exponential-shaped rise to a sigmoidal-shaped rise.

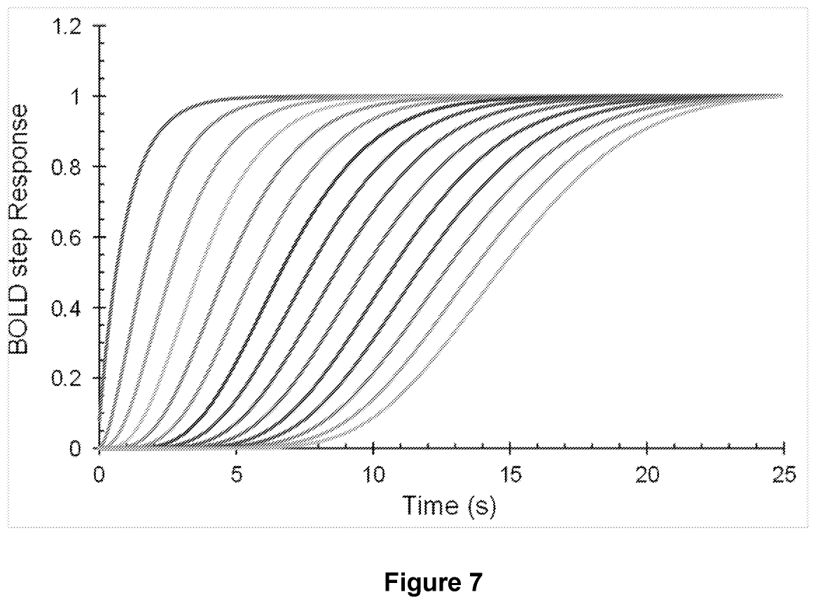

FIG. 7 shows a range of patterns of increase in BOLD signal to a step decrease in [dOHb] derived from the integration of the gamma variate function shown in FIG. 6 and normalized to an amplitude of 1.

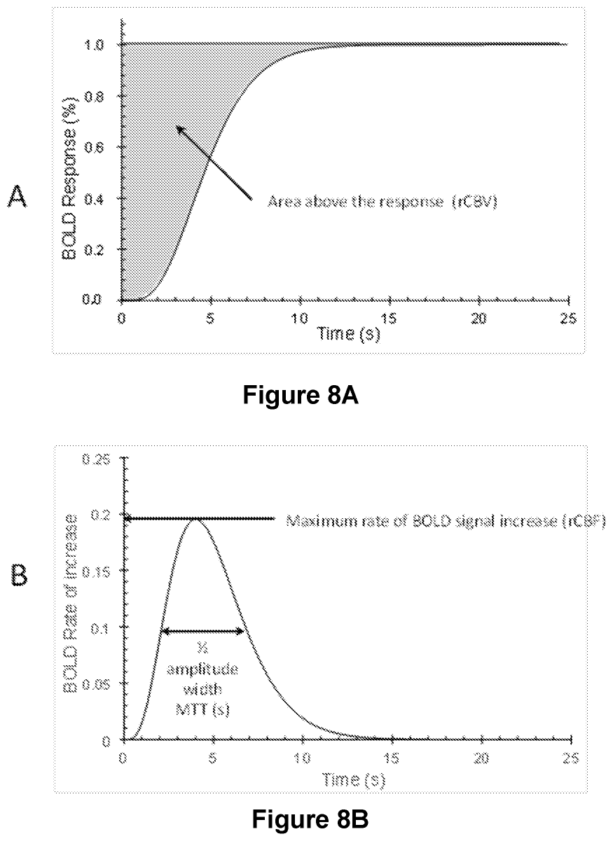

The derivation of the estimates of perfusion measures from the assumed description of the BOLD step response are illustrated in FIG. 8.

FIGS. 8A and 8B show the derivation of the perfusion measures. In FIG. 8A, the relative cerebral blood volume (rCBV) is assumed to be proportional to the area above the step response when scaled to the observed BOLD step response. In FIG. 8B, the relative cerebral blood flow (rCBF) is assumed proportional to the maximum rate of change of BOLD during the step response. Mean capillary transit time (MTT, seconds) is assumed proportional to the half-amplitude width of the BOLD rate of increase vs time gamma variate.

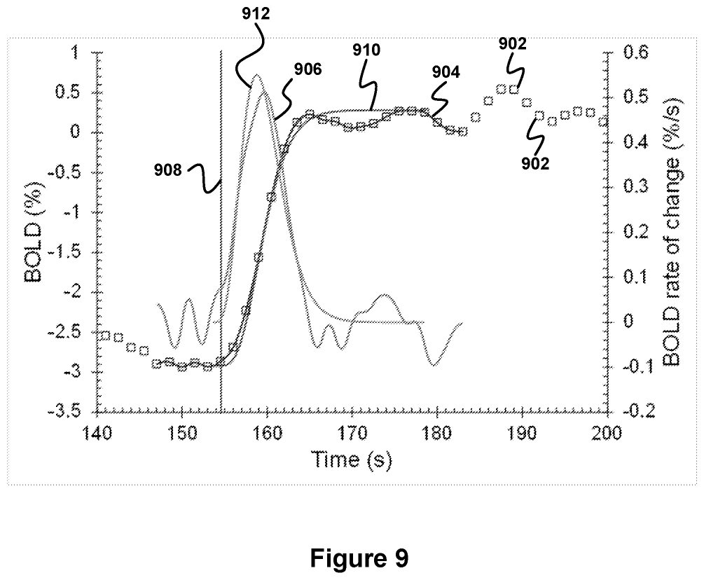

Each voxel BOLD signal is transformed using a series of fitting steps. An example of the fitting process will be described with respect to FIG. 9. Squares 902 indicate the BOLD signal sampled at a TR=1.5 s. Line 904 is a resampling part of this record at dt=0.1 s. Line 906 is the derivative of line 904 showing the rate of change of BOLD during the step response. Line 912 is the gamma variate whose integration yields line 910 fitted to the step change in BOLD.

First, a section of the BOLD signal 25 TR samples long (open square points indicated at 902) is chosen, starting 5 TR before the reference cursor time, and resampled (interpolated) to a dt=0.1 s (line indicated at 904).

Second, the differential of this BOLD signal is then calculated. The differential is represented by line 906.

Third, this differentiated BOLD signal is followed back in time from the maximum to determine the point at which the slope of zero is reached and this time is taken as the start of the step response. The difference of this start time and the reference cursor time (indicated at 908) is the blood arrival time (BAT, s) estimation.

Fourth, the series of BOLD step responses with gamma variate parameter a varied from 1 to 15 in steps of 0.1 as pictured in FIG. 7, is compared with the resampled BOLD step response rate of change (indicated at 904) to find the best correlation.

Fifth, the selected gamma parameter a thus determines the fitted BOLD step response (indicated at 910) and its first derivative (indicated at 912).

Sixth, perfusion measures rCBV, rCBF and MTT are calculated from these fits as shown in FIGS. 8A and 8B.

### 1.6: Discussion

The experimental results show that hemodynamic parameters obtained through the present method are comparable to measures generated using an AIF from a bolus change of gadolinium.

The step stimulus implemented in this experiment provides an actual stimulus that can be directly measured. In contrast, the spike stimulus of the prior art is virtual and must be calculated. The spike stimulus cannot be directly measured, and therefore perfusion metrics obtained using the prior art methods are less accurate.

### Example 2

Testing was undertaken to demonstrate the feasibility of mapping cerebral perfusion metrics with BOLD MRI during modulation of pulmonary venous oxygen saturation.

2.1 Method

The following tests were performed on 7 subjects. 6 of the subjects were healthy volunteers who were non-smokers and not taking any medication. 1 subject had steno-occlusive disease.

An investigational computerized gas blender, (RespirAct™ Thornhill Medical, Toronto, Canada), was used to prospectively target lung PO₂ in the subject while maintaining normocapnia independently of ventilation. All gases and gas sensors were calibrated prior to the study. BOLD images were acquired on a 3 Tesla MRI system (Signa HDx-GE Healthcare™, Milwaukee, WI), with interleaved echo-planar acquisition during end-tidal PO₂ (P_{ET}O₂) manipulation.

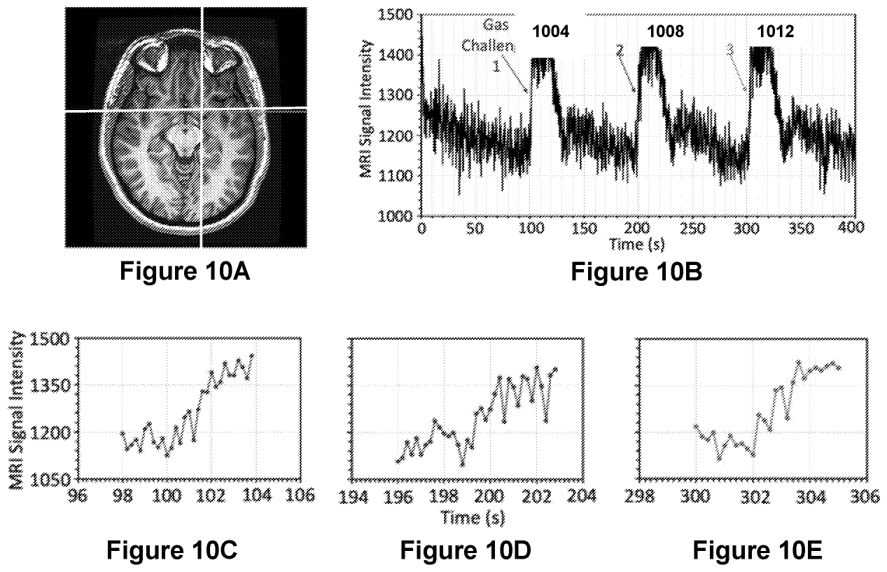

Three different [dOHb] signals were tested, with different temporal profiles. Experiment 1 was performed to evaluate the rapidity of arterial signal changes. The P_{ET}O₂ was lowered to a hypoxic baseline targeted at 40 mmHg for 90 seconds, followed by an increase to 95 mmHg within 1 breath, for 10 second duration, then returning to the hypoxic baseline for another 90 seconds. Normocapnia was maintained throughout all changes in P_{ET}O₂. This challenge was repeated 2 more times. For Experiment 1, MR parameters were TR/TE=200/30 ms, flip angle=30°, and 3-mm isotropic voxels with 3 contiguous slices positioned over the middle cerebral arteries. The selected voxel, which is on or close to the middle cerebral artery, is shown at the crosshairs in FIG. 10A. FIG. 10B shows the MRI signal intensity plotted over time as the SGD device implemented a gas challenge from a hypoxic baseline of 40 mmHg to 95 mmHg within one breath, indicated at 1004. The SGD device maintained P_{ET}O₂ at 95 mmHg for 10 seconds before returning the P_{ET}O₂ to 40 mmHg three consecutive times, indicated at 1008 and 1012. A short TR was chosen to capture the rapidity of arterial signal changes.

The TR of 200 ms provides a high temporal resolution of the signal rise time, reflecting the rapid deoxyhemoglobin changes in the lungs, particularly considering some inevitable dispersion of deoxyhemoglobin in the left atrium and left ventricle. It also illustrates the repeatability of the stimulus. FIGS. 10C to 10E show the high temporal resolution profiles of rise times from the selected voxel. FIG. 10C shows the high temporal resolution profile of rise time for the first gas challenge 1004, FIG. 10D shows the high temporal resolution profile of rise time for the second gas challenge 1008, and FIG. 10E shows the high temporal resolution profile of rise time for the third gas challenge 1012. Fitting a first-order exponential to the rise in the BOLD signal in stimuli 1004, 1008, and 1012 results in time constants of 1.21 s, 1.67 s and 2.10 s, respectively.

In Experiment 2, the same breathing protocol was used but was repeated 4 times in order to assess the degree to which CNR over the whole brain improved as the number of boluses increased. The MR parameters were TR/TE=2000/30 ms, flip angle=85°-, and 3-mm isotropic voxels with 29 contiguous slices.

In Experiment 3, 6 healthy subjects were studied to assess consistency of data between subjects to the extent that the variability of MTT, CBV, and CBF are in the normal physiologic range. In addition, 1 subject was studied to provide a forward-looking perspective of dOHb-calculated perfusion metrics in the presence of steno-occlusive disease. The protocol for this study was the same as Experiment 1 except that the duration of normoxia was extended from 10 s to 20 s to guarantee lung-arterial equilibration at normoxia in a subject with potential lung pathology. The MR parameters were TR/TE=1500/30 ms, flip angle=73°, and 3 mm isotropic voxels, with 29 contiguous slices.

A high-resolution T1-weighted spoiled-gradient-echo sequence was acquired for co-registering the BOLD images and localizing gray matter (GM), white matter (WM), and arterial and venous components. Spoiled-gradient-echo parameters were: 176 slices of 1 mm thick partitions, in-plane voxel size of 0.85×0.85 mm, FOV of 22 cm, and TR/TE=7.88/3.06 msec.

2.2 Conversion of P_{ET}O₂ to [dOHb]

The recorded end-tidal P_{ET}O₂ was converted to SaO₂ using the Hill equation (Equation 1) and normal sea-level physiologic parameter, where n=−4.4921 pH+36.365, K=5E−142 pH157.31, and pH=7.4.

### 2.3 Image Analysis

The image analysis was performed using MatLab 2015a (MathWorks, Natick, MA) and AFNI12 (v19.1.09).

In Experiment 1 (3 slices, TR=200 ms), the data were visually inspected, and 1 voxel with high CNR localized over the middle cerebral artery (MCA) was chosen to analyze the rise time (T) of the BOLD signal using an exponential function model.

In Experiments 2 and 3 (29 slices), standard preprocessing steps including slice time correction and volume re-registration were first applied. Next, SaO₂ was resampled and interpolated to TR intervals and then time-aligned to 1 voxel placed over the MCA. This alignment was carefully chosen to have the onset rise of SaO2 and MCA signal match. The baseline mean (S0) was calculated over ˜30 s prior to the rise of SaO₂. BOLD signal Sₜ was scaled:

$${S}_{c , t} = \frac{{S}_{t} - {S}_{0}}{{S}_{0}} .$$

Subscript t indicates that the variable is a function of time. A TD map was calculated using cross-correlation between S_{c,t}, and multiple SaO₂ curves time shifted from 0 to 7 s by intervals of 0.2 s. The time shift needed to obtain maximum correlation (R) with S_{c,t} was extracted for each voxel to generate a TD.

Sc,t was regressed against the voxel-wise shifted SₐO_{2,t}^{shifted} to calculate the slope of regression, as shown previously in Equation 6. Using the slope of the regression α, the BOLD signal change (ΔBOLD) and CNR were computed according to Equation 7 and 8 (shown above).

### 2.4 Tracking of Contrast in Arteries and Veins

In Experiment 3, arterial and venous voxels were extracted using Δ BOLD, R, and TD. Arterial voxels were defined as all voxels with ΔBOLD>20%, R>0.8, and 0<TD<1.5. Venous voxels were defined as all voxels with ΔBOLD>20%, R>0.8, and 3<TD<5. BOLD time series was averaged within the arterial and venous components.

### 2.5 Relative Perfusion Metrics

Assuming a linear relationship between Sc,t and [dOHb], standard tracer kinetic modeling was used to calculate MTT and rCBV as per Equation 2 (shown above).

β₁ and β₂ account for linear signal drift and baseline, respectively. t represents the residuals. SaO₂ was used as the arterial input function (AIFₜ).

$${R}_{t} = {e}^{- \frac{1}{M T T}}$$

was used for the residue function. It is equal to 1 at time 0 and set to 0 at time equal to 5×MTT. MTT was bound between 1 and 8 s. rCBV and MTT were determined using a least square fitting procedure. rCBF was then calculated using the central volume theorem as rCBF=rCBV/MTT. Notes that rCBV and rCBF were respectively multiplied by 15 and 750 to obtain easily readable values within range of absolutes measures.

### 2.7 Absolute Perfusion Metrics

Assuming that the chosen voxel over the MCA (AIF) contained 100% blood, absolute CBV in mL/100 g was calculated according to Equation 3 (shown above), with tissue density (ρ) equal to 1.04 g/cc and difference in hematocrit in large vessels (kH) equal to 0.73. CBF in mL/100 g/min was then calculated according to Equation 4 (shown above).

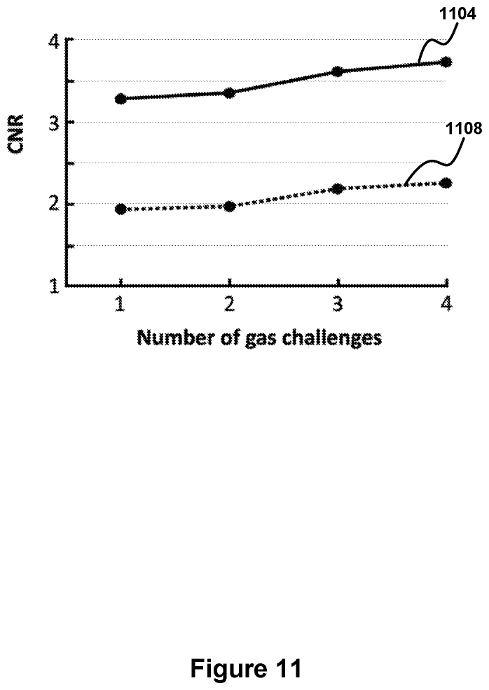

The T1-weighted images were segmented into GM, WM, and CSF using spm8 software. 13 The probability density maps obtained were thresholded at 0.8 to generate a GM and WM masks. One layer of peripheral voxels was eroded from the WM mask. GM and WM masks were used to calculate average values for ΔBOLD signal, CNR, TD, MTT, rCBV and rCBF, and CBV and CBF for all 6 normal controls as well as for the steno-occlusion subject. The average CNR in GM for each of the four gas challenges is shown in FIG. 11 at 1104, and the average CNR in WM for each of the four gas challenges is shown in FIG. 11 at 1108.

### 2.8 Results

The hypoxic PaO₂ at baseline was sensed by some subjects as a vague shortness of breath; however, they did not find it very challenging to maintain PO₂ of 40 mmHg for 90 s, nor were they able to identify the transition to hypoxia or any sense of relief from a return to normoxia. FIGS. 10A-C illustrate the signal in middle cerebral artery displaying a minimal extent of dispersion of the paramagnetic bolus shape resulting from the lung itself, the confluence of the pulmonary veins, the wash-in and wash-out of the blood in the left ventricle, and any mixing in the aorta and extracranial arteries. It demonstrates that this technique has the ability to induce rapid changes in BOLD signal during the transition from hypoxia to normoxia on the order of a few seconds. There is also remarkable consistency in the successive gas challenges and resulting BOLD signal changes, with little drift (FIG. 10B). The measured rise-time (T) for each bolus was 1.21 s, 1.67 s, and 2.10 s.

In Experiment 2, FIG. 11 shows that after 4 gas boluses CNR was 3.7 in GM and 2.3 in WM. Averaging results of gas challenges resulted in minimal improvement of CNR.

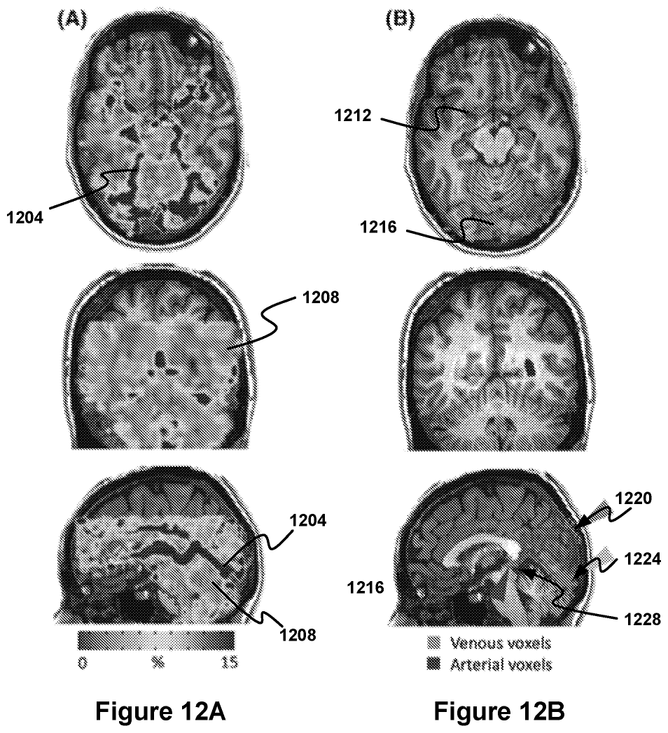

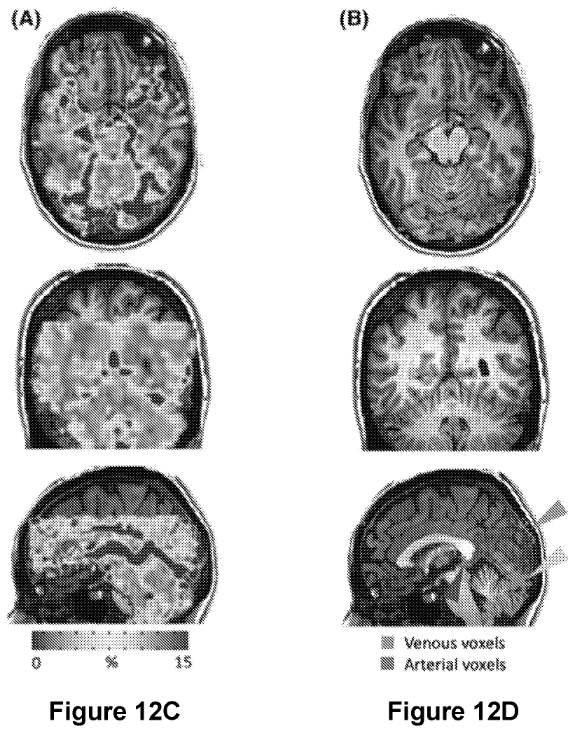

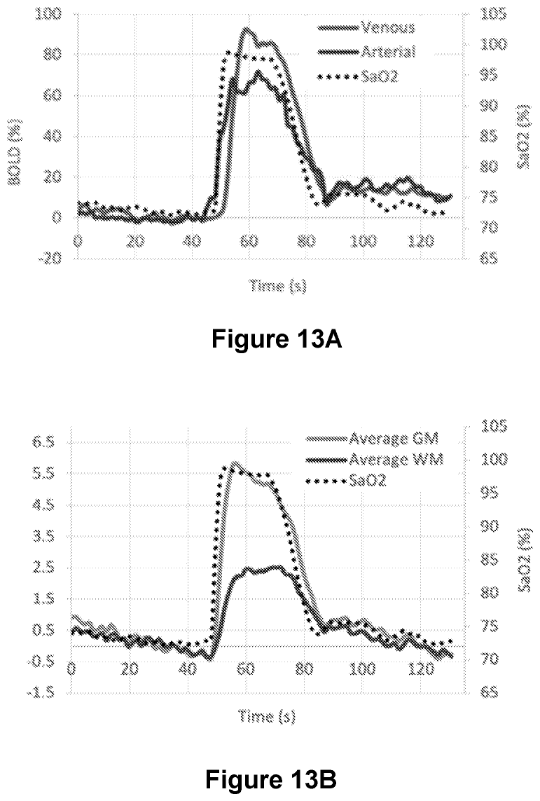

FIGS. 12 and 13 represents an example from the healthy subjects in Experiment 3.

FIGS. 12A and 12B are grayscale versions of FIGS. 12C and 12D.

FIG. 12A (and FIG. 12C) shows maps of magnitude of BOLD signal change according to the accompanying color scale. Red (1204) indicates 15% BOLD signal change and blue (1208) indicates 0% BOLD signal change.

In FIG. 12B (and FIG. 12D), high signal voxels sorted according to TD and magnitude of Δ BOLD. The chosen criteria for identifying the arterial component were ΔBOLD>20%, R>0.8, and TD<1.5 s. The criteria for the venous component were ΔBOLD>20%, R>0.8, and 3<TD<5 s. Late filling voxels colocalized with known large sinovenous structures: superior sagittal sinus (green arrow, 1220), straight sinus (yellow arrow, 1224), and great vein of Galen (blue arrow, 1228).

FIG. 12B illustrates that the distribution of early arriving large amplitude voxels (colored red and indicated at 1212) is consistent with the locations of middle and anterior cerebral arteries; and the later-arriving large amplitude changes (colored blue and indicated at 1216) are consistent with the location of the major veins and venous sinuses.

FIG. 13A is a graph showing bolus arrival times. The SaO₂ curve was calculated from end-tidal PO₂ data and synchronized with the rise in arterial BOLD signal. FIG. 13A shows the corresponding average BOLD signal over arterial and venous voxels separately. FIG. 13B is a graph showing the SaO2 time series, as well as the average GM and WM BOLD signal. FIG. 13B shows the average GM and WM BOLD time series with respect to calculated SaO₂. Average BOLD signal in GM is about twice as large as in WM (Table 1).

TABLE 1

| | GM | WM | GM/WM |
|---|---|---|---|
| BOLD (%) | 6.3 ± 1.2 | 2.5 ± 0.6 | 2.5 |
| TD (s) | 2.3 ± 0.6 | 3.6 ± 0.7 | 0.6 |
| CNR | 4.3 ± 1.3 | 2.6 ± 0.7 | 1.7 |
| MTT (s) | 3.9 ± 0.6 | 5.5 ± 0.6 | 0.7 |
| rCBV | 3.7 ± 0.9 | 1.6 ± 0.4 | 2.3 |
| rCBF | 70.1 ± 8.3 | 20.6 ± 4 | 3.4 |
| CBV (mL/100 g) | 4.1 ± 0.9 | 1.8 ± 0.5 | 2.3 |
| CBF (mL/100 g/min) | 97.2 ± 18.7 | 28.7 ± 5.9 | 3.4 |

FIG. 12A shows BOLD signal change clearly distinguished large vessels, GM, and WM. All group hemodynamic data are presented in Table 1, with the last column being the ratio of GM over WM.

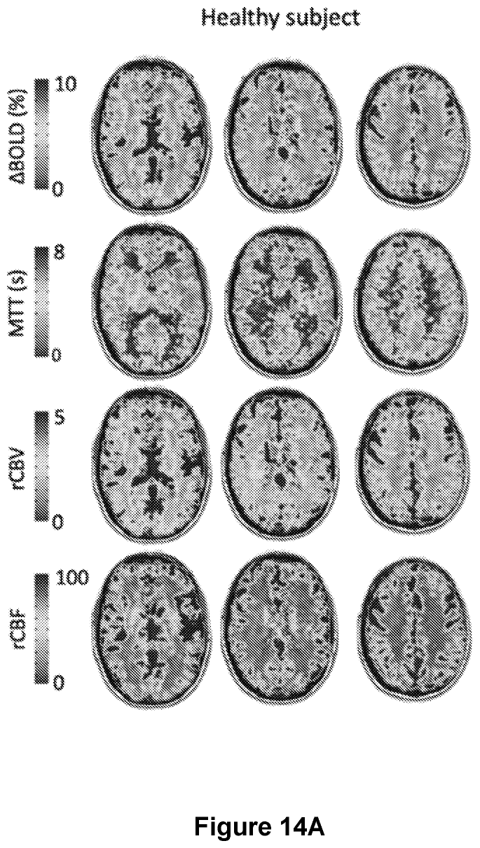

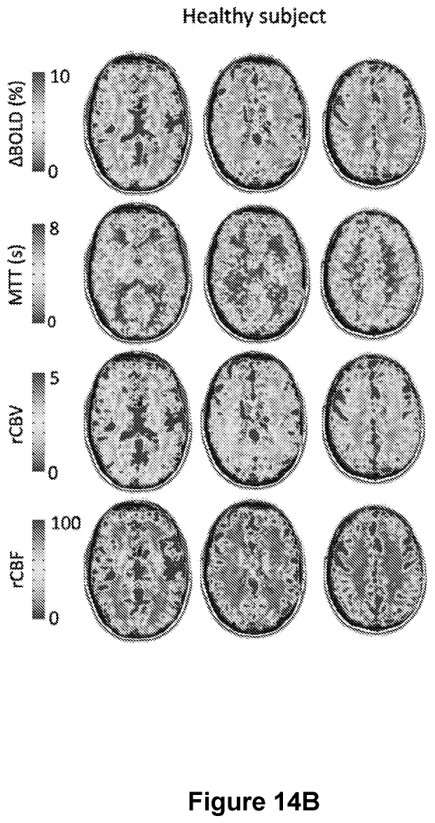

FIGS. 14A and 14B show axial brain slices of ΔBOLD, MTT, rCBV, and rCBF for the same healthy subject as in FIGS. 12 and 13. FIG. 14A is a grayscale version of FIG. 14B. Each row represents 3 axial slices for 1 metric: ΔBOLD, MTT, rCBV, and rCBF.

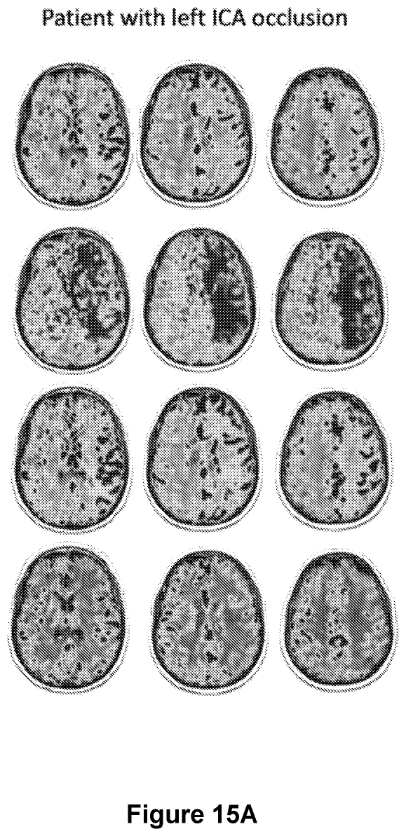

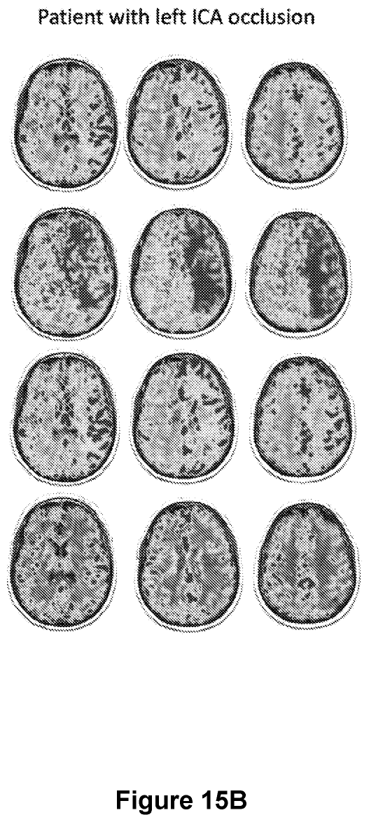

FIGS. 15A and 15B show the same for the subject with left internal carotid stenosis. FIG. 15A is a grayscale version of FIG. 15B. Each row represents 3 axial slices for 1 metric: ΔBOLD, MTT, rCBV, and rCBF. FIG. 15 shows an elevated rCBV, prolonged MTT, and diminished rCBF on the left side of the brain with the stenosis. For the healthy subject shown in FIG. 14, all perfusion metrics show a symmetrical pattern between left and right hemisphere of the brain.

### 2.9 Discussion

This is the first report confirming the proof-of-principle by Vu et al. (Vu C, Chai Y, Coloigner J, et al. Quantitative perfusion mapping with induced transient hypoxia using BOLD MRI. Magn Reson Med. 2021; 85:168-181) that abrupt perturbations in endogenous [dOHb] can be used as a vascular contrast agent to perform dynamic susceptibility contrast imaging and cerebral perfusion quantification. Our work extends this study by implementing a repeatable, precisely targeted isocapnic abrupt change in [dOHb] as an arterial blood susceptibility contrast agent, enabling a direct measure of an arterial input function.

Rapid transitions from hypoxic baseline to full oxygenation (no [dOHb]) are achievable within a single breath (FIG. 10B). This rapid reduction in [dOHb] is reflected in an abrupt increase in BOLD signal with high CNR throughout the arterial and venous compartments of the brain (FIG. 13A). The rapidity of the change in BOLD implies little dispersion of the bolus prior to arrival into the brain vasculature.

The degree of dispersion observed would compare favorably to that of intravenously injected contrast agents because it eliminates an entire pathway over which dispersion can develop, including the brachial vein, subclavian vein, right atrium, and right ventricle. Rapid contrast generation in the lung bypasses this segment of the dispersive pathway, limiting dispersion to passage within the left atrium and left ventricle, with the latter dispersion depending on the left ventricular ejection fraction. The respiratory source of contrast generation therefore enables an arterial contrast bolus as close to a square wave input function as possible, short of direct contrast injection into large arteries.

As expected, the rise in the arterial signal precedes the rise in the venous signal. The larger rise in the venous BOLD signal is consistent with a lower baseline of the unscaled BOLD signal. The reason for this is that large veins/sinuses are bigger than large arteries and more likely to contain an imaging voxel with 100% blood that has high [dOHb], and therefore low signal, when compared to voxels over arteries that include both arterial blood and adjacent tissue that has roughly 2% to 6% blood volume.

Therefore, when scaling the image, dividing by the lower baseline in veins yields a higher percent change in BOLD signal over veins. Importantly, the present study demonstrates repeatability of the method in a single subject (Experiment 1 and 2), as well as between subjects (Experiment 3).

In this report, the relationship between BOLD signal and oxygen saturation was assumed to be linear. However, although the extravascular signal depends linearly on oxygen saturation, the intravascular BOLD signal depends quadratically on oxygen saturation. As a result, CBV values may be underestimated in voxels with high CBV values. 14 For any DSC experiment (gadolinium- or dOHb-based), the scaling constant between tissue R₊₂ and the contrast agent represents a complicated function of vascular geometry and proton mobility (at the subvoxel level), making absolute quantification challenging.

Fortunately, relative perfusion mapping is sufficient for most clinical applications. The assumption of a mono-exponential residue function is a simplification that may be violated in some diseased states but should be appropriate to use in healthy subjects. However, this model might be less sensitive to noise considering that the CNR in the dOHb technique is several folds smaller than using injection of gadolinium. Other analysis methods may be possible including bi-exponential residue function and model-free techniques.

SaO₂ has been used in place of a traditional arterial input function (AIF) derived from the BOLD signal on a cerebral vessel. This eliminates the disadvantages caused by nonlinearities of the BOLD signal as related to voxel size, volume averaging of the voxel between vessel and adjacent tissue, and nonlinearities of the magnetic field at the intra- and extravascular interface. Because the shape and size of the AIF determines MTT and CBV, results might be quite heterogenous depending on the choice of voxel as the AIF. In this context, using SaO₂ as an input function yields more reproducible results.

Quantitative perfusion metrics were also calculated using an AIF from within the middle cerebral artery to scale rCBV. The SDs for absolute CBV and CBF are reasonably small. The averages appear to be overestimated. As voxels along the MCA are not certain to be fully contained inside the vessels and are contaminated with surrounding tissue voxels, the associated scaling factor to calculate CBV may overestimate true values. This issue may be resolved with improved spatial resolution.

Induction of hyperoxia has been used to induce BOLD signal changes to enable measurement of CBF, CBV, and MTT.15 The signal characteristics with a hyperoxic challenge differ from intravascular paramagnetic contrast injection and from hypoxic challenge as in the present method. The hyperoxic inhalation does not change the [dOHb] in the arteries; rather, it simply increases oxygen dissolved in the plasma. On arrival to the tissues, the excess oxygen dissolved in the plasma spares that bound to hemoglobin. As opposed to our method, this mechanism cannot generate a direct arterial signal change.

Hypoxia has been previously used by other groups to calculate CBV.16 However, a very mild hypoxic stimulus was used in which oxygen saturation was only dropped to ˜95%, generating ˜1% BOLD change with substantial noise. In the present method, the average SaO₂ dropped to 70%, with a ˜6% BOLD signal change in GM and a contrast to noise ˜4.

It is possible that profound hypoxia may itself affect CBF. If hypoxia stimulates the hypoxic ventilatory response, hypocapnia is avoided because the RespirAct™ is uniquely capable of maintaining normocapnia. Under these circumstances, CBF is likely unaffected. Hypoxia should not result in substantial increases in CBF if PO₂ is maintained greater than 40 mmHg17; if this is to occur, the response is likely delayed beyond our brief observation period.

Vu et al. reported the generation of dOHb as a contrast agent by the administration of nitrogen as a hypoxic gas in control subjects as well as subjects with chronic anemia syndromes. This stimulus caused transient increases in dOHb with temporal dynamics similar to bolus gadolinium injections, allowing quantification of CBV, CBF, and MTT over a broad range, corroborated by phase contrast and arterial spin labeling. Although clearly establishing feasibility, that initial study suffered two key limitations. Firstly, nitrogen influx produced transient tachypnea and hypocapnia that would interact with CBF. Secondly, the washout of room air with the introduction of nitrogen into the gas reservoir of the apparatus introduced an equipment delay in transition that compounded an additional delay required for the wash out of oxygen from the subject's lungs before the dOHb in the blood could change. In our study, the declines in [dOHb} with re-saturation were reliably controlled and rapidly implemented as shown by their precise repeatability and implementation within 1 or 2 breaths, thus enabling the development of a predictable and measurable sharp leading edge of the AIF.

The reductions of PaO₂ to 40 mmHg in our study were uncomfortable to some subjects but tolerable. Some subjects found the low PaO₂ at baseline was undetectable in the sense that they were unable to discern normoxic from hypoxic gas, causing no distress. Within this small cohort, the hypoxic stimulus was not excessively challenging. A paradigm with a normoxic baseline and transient hypoxic stimulus, and/or a reduction of PaO₂ to only 50 mmHg, is expected to be even better tolerated.

Finally, the exposure to brief periods of hypoxia in our study is not considered harmful for healthy subjects. For example, in La Paz, the capital city of Bolivia at 4000 m elevation, most of the population as well as visitors have PO₂ in the range of 50-60 mmHg, which may be associated with some limitation in exercise tolerance but not serious illness. For further perspective, most of the human population undergoes multiple (5-25) episodes of apnea (cessation of breathing) during sleep. PaO2 has frequently been observed to fall to 30-40 mmHg. Furthermore, exposure to SaO2 lower than 75% for durations up to 30 min have shown no adverse effects.19 Therefore, limited interrupted exposure to 75% SaO2 is within safety margins.

In this study, hypoxia was established as a baseline, and a normoxic lung changes with decreasing [dOHb} was used as the stimulus. The same imaging principles would apply in the reverse situation with a normoxic baseline and hypoxic gas challenges with increasing [dOHb]. This latter approach would further reduce any safety concerns in some subjects with comorbidities. The hypoxic baseline was chosen for the present experiments because the conditions for rapid transition to normoxia were more readily attained at the time of the study than the reverse. Implementation of the study using normoxia as the baseline may produce similar results.

The recruitment of endogenously generated dOHb by precise, reproducible, and tolerable stimuli generate large susceptibility signals and thereby offers the potential for clinical translation.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A method comprising:
targeting a first partial pressure of oxygen in arterial blood (PaO₂) in a subject using a sequential gas delivery device for a first duration of time;
targeting a second PaO₂ using the sequential gas delivery device for a second duration of time;
measuring a blood oxygen level dependent (BOLD) signal in a voxel of the subject's brain using magnetic resonance imaging (MRI) while targeting the first and second PaO₂;
calculating a rate of change for the BOLD signal as the first PaO₂ transitions to the second PaO₂;
fitting the rate of change to a gamma variate function;
calculating an integral of the gamma variation function; and
calculating a perfusion metric for the voxel based on the gamma variate or the integral of the gamma variate function.

2. A method comprising:
targeting a first partial pressure of oxygen in arterial blood (PaO₂) in a subject using a sequential gas delivery device for a first duration of time;
targeting a second PaO₂ using the sequential gas delivery device for a second duration of time;
measuring a magnetic resonance signal in a voxel of the subject's brain using magnetic resonance imaging (MRI) while targeting the first and second PaO₂;
calculating a rate of change for the magnetic resonance signal; and
calculating a perfusion metric for the voxel based on the rate of change and an integral of the rate of change.

3. The method of claim 2 wherein the first PaO₂ is lower than the second PaO₂.

4. The method of claim 3 wherein the first PaO₂ corresponds to hypoxia in the subject and the second PaO₂ corresponds to normoxia in the subject.

5. The method of claim 4 the first PaO₂ about 40 mmHg and the second PaO₂ is about 95 mmHg.

6. The method of claim 5 further comprising maintaining normocapnia while targeting the first PaO₂.

7. The method of claim 2 wherein the sequential gas delivery device is programmed to target the first PaO₂ within one breath.

8. The method of claim 2 wherein the sequential gas delivery device is programmed to target the second PaO₂ within one breath.

9. The method of claim 2 wherein the perfusion metric is selected from a group consisting of: contrast arrival time (AT), relative mean transit time (MTT), relative cerebral blood flow (rCBF), and relative cerebral blood volume (rCBV), absolute CBV, absolute MTT, and absolute CBF.

10. The method of claim 2 wherein the magnetic resonance signal is measured using blood oxygen level dependent (BOLD) MRI.

11. The method of claim 2 further comprising fitting the rate of change to a gamma variate function, wherein calculating the perfusion metric is based on the gamma variate function.

12. The method of claim 11 further comprising calculating the integral of the gamma variate function, wherein calculating the perfusion metric is based on the integral of the gamma variate function.
